(19) United States
(12) Patent Application Publication
(10) Pub. No.: US 2026/0263497 A1
(43) Pub. Date: Sep. 10, 2026

(54) METHOD OF TREATING SCHIZOPHRENIA AND COMPOSITION FOR USE THEREIN

(71) Applicant: Academia Sinica, Taipei (TW)

(72) Inventors: Yijuang CHERN, Taipei City (TW); Wen-Sung LAI, Taipei City (TW); Jim-Min FANG, Taipei City (TW)

(73) Assignee: Academia Sinica, Taipei (TW)

(21) Appl. No.: 18/881,373

(22) PCT Filed: Jul. 7, 2023
(86) PCT No.: PCT/US2023/027098

§ 371 Date: Jan. 6, 2025

Related U.S. Application Data
(60) Provisional application No. 63/358,948, filed on Jul. 7, 2022.

(51) Int. Cl. A61K 31/7076; A61P 25/18

(52) U.S. Cl. CPC A61K 31/7076; A61P 25/18

(57) ABSTRACT

A method of treating schizophrenia is provided, including administrating to a subject a compound of formula (I), (II) or (III): (I) (II) (III) or a pharmaceutically acceptable salt thereof, wherein X is halogen. A composition for use in a method of treating schizophrenia, including administering to a subject in need thereof the composition comprising a compound of formula (I), (II) or (III) as shown above is also provided.

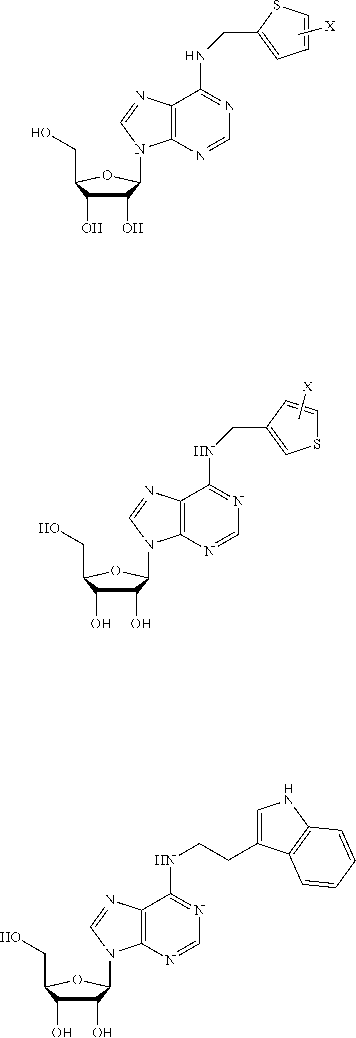

### CROSS REFERENCE TO RELATED APPLICATIONS

This application is the National Phase of PCT International Application No. PCT/US2023/027098, filed on Jul. 7, 2023, which claims priority under 35 U.S.C. 119(e) to U.S. Provisional Application No. 63/358,948, filed on Jul. 7, 2022, all of which are hereby expressly incorporated by reference into the present application.

### FIELD OF INVENTION

The present disclosure relates to a method of treating schizophrenia and a composition for use in a method of treating schizophrenia.

### BACKGROUND OF THE INVENTION

Schizophrenia is a severe mental illness characterized by extensive brain dysfunctions, which includes distortions in thinking, perception, emotions, language, sense of self and behavior. This devastating disease counts for ~1% prevalence in the population, and tends to develop in early adulthood and can persist throughout the patient's lifetime.¹

Alterations in dopaminergic signaling have been implicated in schizophrenia and amphetamine addiction. Dopamine hyperactivity has been the predominant pathophysiologic hypothesis of schizophrenia (i.e., the dopamine hypothesis of schizophrenia). Hyperactive dopaminergic signal transduction is thought to be closely related to the symptoms associated with schizophrenia. Pharmacological modification of dopamine transmission therefore has long been employed as a therapeutic tool.² Previous positron emission tomography (PET) studies revealed that dopaminergic functioning is dysregulated at the prodromal phase of patients with schizophrenia, and further worsens along the development of psychosis.³ Although the risk genes for schizophrenia are linked to the upstream and downstream pathways of the dopaminergic system, none is directly involved in the synthesis and release of dopamine. Vulnerability of dopaminergic neurons to environmental and developmental risk factors are attributed to these upstream factors, while the effects of dysregulation are further amplified by the downstream factors.⁴ To date, antipsychotics that modulate dopamine neurotransmission remain to be the major class of drugs used to treat schizophrenia.⁵ However, about one third of patients do not respond to dopaminergic antipsychotics. In addition, the use of antipsychotics may result in many unwanted adverse effects, such stiffness, shakiness, akathisia, tardive dyskinesia, sexual problems, sleepiness, weight gain etc. Besides, the efficacy of currently available antipsychotics towards treatments of negative and cognitive symptoms of schizophrenia is limited.⁵ Thus, searching for novel therapeutic targets and developing new drugs with fewer side effects for the mental diseases are timely and critical.

### SUMMARY OF THE INVENTION

Based on the above reasons, the present invention provides a new method of treating schizophrenia by using the compounds of adenosine analogues for lowering side effects and/or treating schizophrenia-related symptoms.

In an aspect of the present invention, a method of treating schizophrenia, including administering to a subject in need thereof a compound of formula (I), (II) or (III):

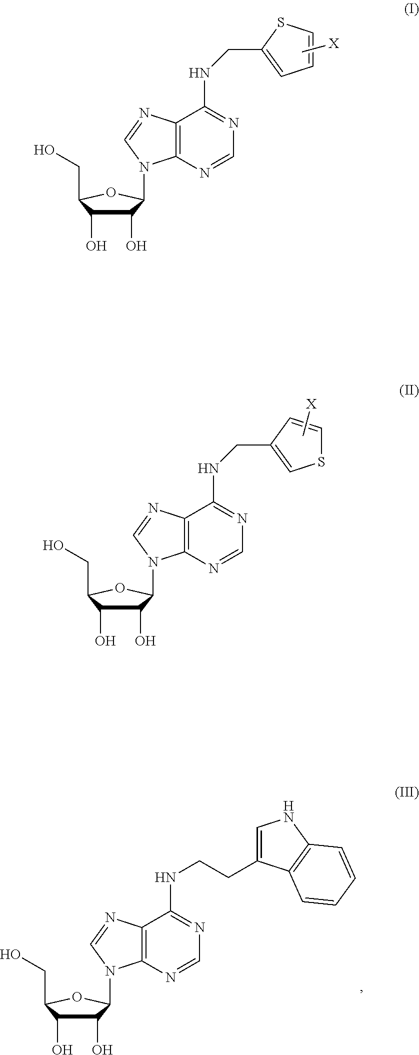

a pharmaceutically acceptable salt thereof, or a composition thereof, wherein X is halogen.

In another aspect of the present invention, a composition for use in a method of treating schizophrenia, including administering to a subject in need thereof the composition including a compound of formula (I), (II) or (III) as shown above.

Preferably, the compound is selected from the group consisting of N⁶-[(3-halothien-2-yl)methyl]adenosine, N⁶-[(4-halothien-2-yl)methyl]adenosine, and N⁶-[(5-halothien-2-yl)methyl]adenosine. More preferably, the compound is selected from the group consisting of N⁶-[(5-bromothien-2-yl)methyl]adenosine, N⁶-[(4-bromothien-2-yl)methyl]adenosine, N⁶-[(3-bromothien-2-yl)methyl]adenosine, N⁶-[(5-chlorothien-2-yl)methyl]adenosine, N⁶-[(4-chlorothien-2-yl)methyl]adenosine, and N⁶-[(3-chlorothien-2-yl)methyl]adenosine.

Preferably, the compound is selected from the group consisting of N⁶-[(2-halothien-3-yl)methyl]adenosine, N⁶-[(4-halothien-3-yl)methyl]adenosine, and N⁶-[(5-halothien-3 -yl)methyl]adenosine. More preferably, the compound is selected from the group consisting of N⁶-[(2-bromothien-3-yl)methyl]adenosine, N⁶-[(4-bromothien-3-yl)methyl]adenosine, N⁶-[(5-bromothien-3-yl)methyl]adenosine N⁶-[(2-chlorothien-3-yl)methyl]adenosine, N⁶-[(4-chlorothien-3-yl)methyl]adenosine, and N^{6z-[(}5-chlorothien-3-yl)methyl]adenosine.

Preferably, the compound, a pharmaceutically acceptable salt thereof, or a composition thereof is administered by an oral, intravenous, intramuscular, subcutaneous or intraperitoneal route.

Preferably, the composition further includes a pharmaceutically acceptable carrier, excipient or vehicle.

Therefore, the present invention at least provides the following advantages:
1. The claimed compounds are orally active adenosine analogues that can pass the blood-brain-barrier. Thus, the method of the present invention can be easily performed by administering the claimed compounds to a subject via an oral route.
2. The claimed invention has potential to suppress the hyperlocomotor response induced by excessive dopamine

### BRIEF DESCRIPTION OF THE DRAWINGS

FIG. 1A illustrates the effect of JMF3464 alone on locomotor activity for 1 hour with one-way ANOVA of total traveled distance, wherein * p<0.05 and *** p<0.001; data are presented as the mean f SEM.

FIG. 1B illustrates the effect of JMF3464 alone on locomotor activity with two-way ANOVA of traveled distance in 5-minute time bins, wherein * p<0.05 and *** p<0.001; data are presented as the mean f SEM.

FIG. 2 illustrates the effect of (A) JMF3464 and (B) CGS21680 on methamphetamine (METH)-induced hyperlocomotor activity for 1 hour wherein *p<0.05 and *** p<0.001; data are presented as the mean f SEM.

FIG. 3A illustrates the effect of JMF3464 on the METH-induced hyperlocomotor activity in 5-minute time bins, wherein * p<0.05 and *** p<0.001; data are presented as the mean f SEM.

FIG. 3B illustrates the effect of CGS21680 on the METH-induced hyperlocomotor activity in 5-minute time bins, wherein * p<0.05 and *** p<0.001; data are presented as the mean f SEM.

FIG. 4A illustrates the effect of 1 mg/kg JMF3464 on METH-induced condition place preference at preconditioning and postconditioning phases, wherein, * p<0.05, ** p<0.01 and *** p<0.001; data are presented as the mean f SEM.

FIG. 4B illustrates the effect of 3 mg/kg of JMF3464 on METH-induced condition place preference at preconditioning and postconditioning phases, wherein, * p<0.05, ** p<0.01 and *** p<0.001; data are presented as the mean f SEM.

FIG. 5 illustrates the effect of JMF 1907 on METH-induced hyperlocomotor activity in an open field test for 1 hour wherein * p<0.05 and *** p<0.001; data are presented as the mean f SEM.

FIG. 6 illustrates the effect of JMF 1907 on the METH-induced hyperlocomotor activity in an open field test in 5-minute time bins; data are presented as the mean of each group at each 5-minute time bins.

### DETAILED DESCRIPTION

Given that adenosine and dopamine interact antagonistically in living mammals and that antagonistic interactions between adenosine receptor and dopamine receptor in neurons and astrocytes, the inventors of the present invention researched whether controlling the adenosinerigic pathway may modulate the abnormality of dopaminergic system in schizophrenia, and proposed that stimulation of adenosine signaling pathway may indirectly suppress the hyperfunction of dopamine receptors in schizophrenia and relieve the symptoms of schizophrenia. Delineating the downstream molecular pathway of adenosine and dopamine interaction is also critical for searching for new therapeutic targets.

Methamphetamine (METH) is a potent dopamine transporter inhibitor that markedly increases extracellular dopamine levels in the brain. METH, a potent psychostimulant, can induce psychosis among recreational and chronic users, whose psychotic syndrome shows similarities to schizophrenia. METH-induced psychosis symptomatology has been described as indistinguishable from that of schizophrenia. Thus, the present invention aims at dissecting the molecular cross-talk between dopamine and adenosine receptors in the striatum, which is one of critical brain regions for dopamine pathways and schizophrenia, and taking advantage of METH to evaluate the in vivo efficacy of the claimed compounds, new adenosine analogues, in a mouse model of METH-induced psychosis.

In one embodiment, a method of treating schizophrenia is provided, including administrating to a subject a compound of formula (I), (II) or (III):

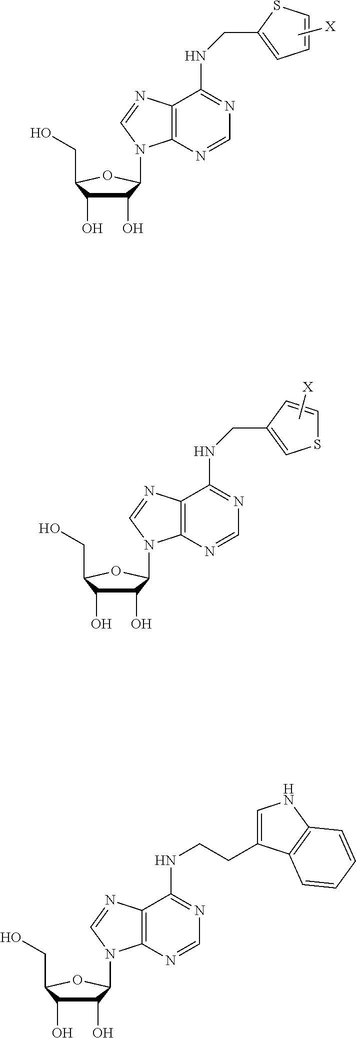

a pharmaceutically acceptable salt thereof, or a composition thereof, wherein X is halogen. Wherein, the compound of formula (III) is also called “JMF 1907” herein.

In another embodiment, the compound may be selected from N⁶-[(3-halothien-2-yl)methyl]adenosine, N⁶-[(4-halothien-2-yl)methyl]adenosine, and N⁶-[(5-halothien-2-yl)methyl]adenosine. Preferably, the compound is N⁶-[(5-bromothien-2-yl)methyl]adenosine (also called “JMF3464”), N⁶-[(4-bromothien-2-yl)methyl]adenosine, N⁶-[(3-bromothien-2-yl)methyl]adenosine, N⁶-[(5-chlorothien-2-yl)methyl]adenosine (also called “JMF3818”), N⁶-[(4-chlorothien-2-yl)methyl]adenosine, N⁶-[(3-chlorothien-2-yl)methyl]adenosine, or a commination thereof.

In another embodiment, the compound may be selected from N⁶-[(2-halothien-3-yl)methyl]adenosine, N⁶-[(4-halothien-3-yl)methyl]adenosine, and N⁶-[(5-halothien-3-yl)methyl]adenosine. Preferably, the compound is N⁶-[(2-bromothien-3-yl)methyl]adenosine, N⁶-[(4-bromothien-3-yl)methyl]adenosine, N⁶-[(5-bromothien-3-yl)methyl]adenosine N⁶-[(2-chlorothien-3-yl)methyl]adenosine, N⁶-[(4-chlorothien-3-yl)methyl]adenosine, or N⁶-[(5-chlorothien-3-yl)methyl]adenosine, or a commination thereof.

In one embodiment, the compound, a pharmaceutically acceptable salt thereof, or a composition thereof is administered by an oral, intravenous, intramuscular, subcutaneous or intraperitoneal route.

### EXAMPLES

### Example 1

### Evaluation Effect of Adenosine Analogue JMF3464 on Schizophrenia

N6-[(5-bromothien-2-yl)methyl]adenosine, also called “JMF3464” and having the structure as follows, is a small adenosine analogue. To assess the beneficial potential of JMF3464, the present invention determined the effect of JMF3464 in vivo using several behavioral tasks, including the METH-induced behavioral sensitization and the conditioned place preference (CPP).

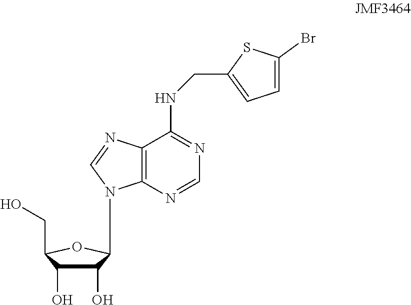

### Methods and Results

Animals: Male mice (C57BL/6; 2-3 months) were purchased from and maintained in the animal facility of National Taiwan University (Taipei, Taiwan) under standard condition with a 12-hr light/12-hr dark cycle. All animal experimental procedures were performed in accordance with the guidelines established by the Institutional Animal Care and Use Committee (IACUC) of the National Taiwan University and Institute of Biomedical Sciences (IBMS) at Academia Sinica (Taipei, Taiwan).

The locomotor activity: The locomotor activity was monitored and recorded using SMART 3.0 video tracking software (Panlab Harvard Apparatus, Barcelona, Spain) for 1 hour following the manufacture's protocol.

### Effect of JMF3464 on Locomotor Function

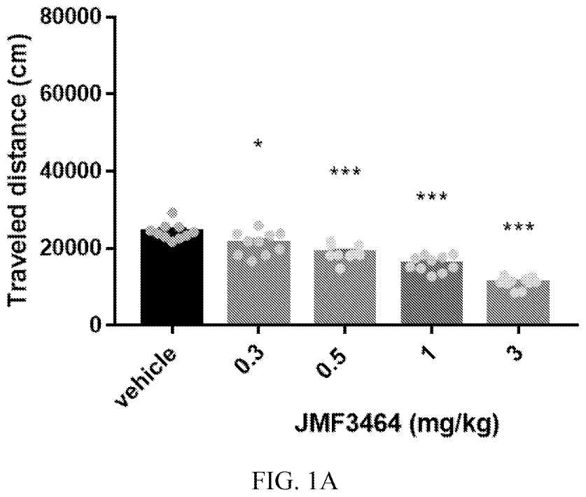

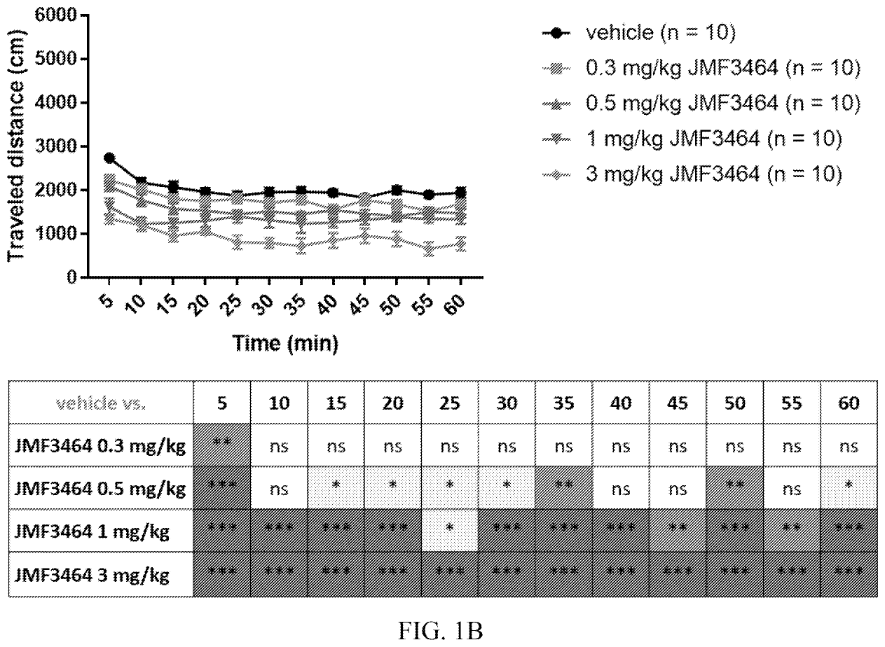

Male drug-naïve WT mice (3-6 months old) received a single dose JMF3464 injection (0.3, 0.5, 1, and 3 mg/kg, respectively) via intraperitoneal injection and the locomotor activity was recorded immediately after injection. A vehicle group is used as control group. The data were expressed and analyzed as one-way ANOVA of total travelled distance for 60 minutes (see FIG. 1A) and two-way ANOVA of distance by 5-minute time bin (see FIG. 1B). As shown in FIGS. 1A and 1B, the injection of JMF3464 alone showed a dose-dependent effect on locomotor activity, and resulted in a slight reduction (~20%) of total locomotor activity that occurred at all 5-minute time bins.

### METH-Induced Behavioral Sensitization

Methamphetamine (METH) is a dopamine enhancer that elevates synaptic dopamine in the brain and is frequently used to induce schizophrenia-like behaviors in mice.⁶ Thus, METH-induced psychosis mice was used to evaluate the in vivo efficacy of JMF3464.

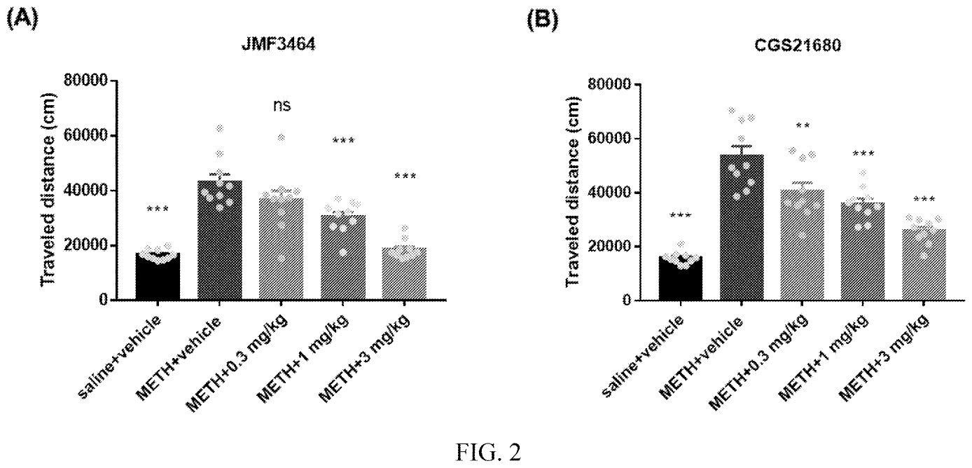

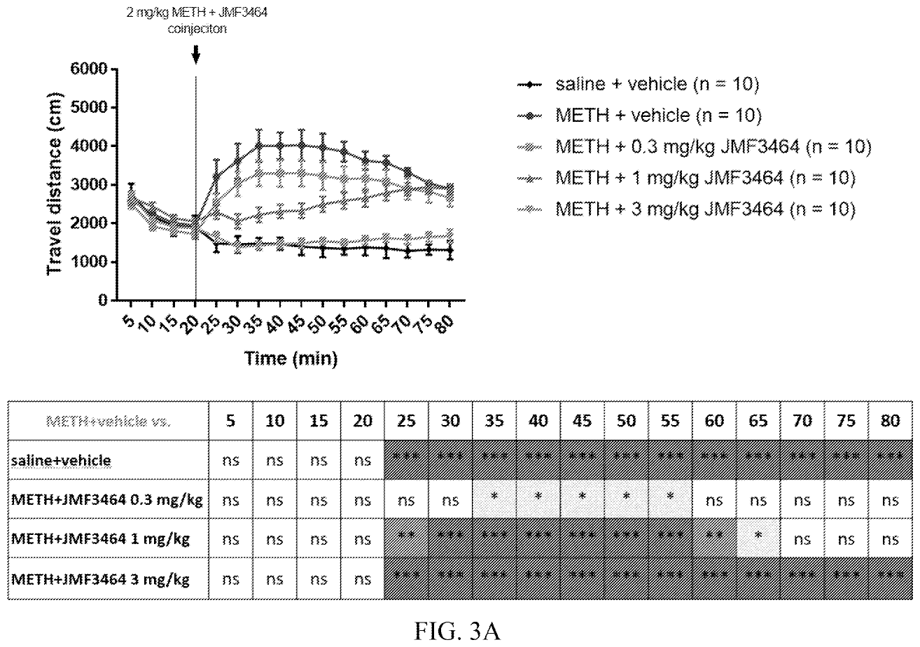

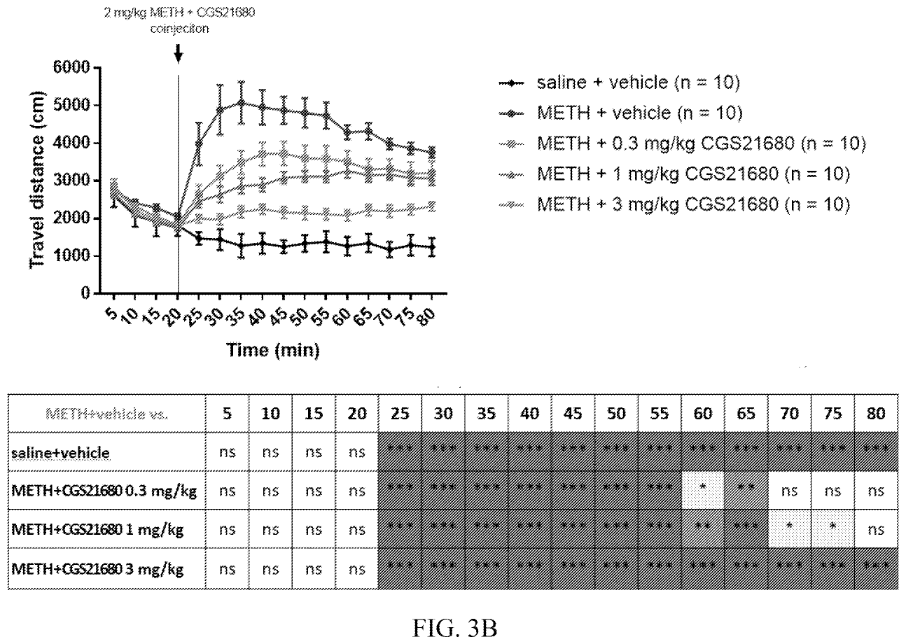

Male METH-naïve wild-type (WT) mice (3-6 months old) received intraperitoneal co-injections of JMF3464 (0.3, 1, or 3 mg/kg) and METH (2 mg/kg) immediately after establishing a 20-minute baseline for locomotor activity. The locomotor activity was recorded for 60 minutes after injection. A saline+vehicle group and a METH+vehicle group are used as control groups. In addition, under the same experimental conditions as JMF3464, the compound CGS21680 (A2A receptor agonist) with potential for treating schizophrenia was used as a comparison. The resulted data was expressed and analyzed as one-way ANOVA of total travelled distance (see FIG. 2) and two-way ANOVA of distance by 5-minute time bin (see FIGS. 3A and 3B) and.

As shown in FIG. 2, compared to the control group (saline+vehicle group), administering METH (2 mg/kg, METH+vehicle group) to mice induced approximately 200% hyperlocomotion. Moreover, co-treatment with JMF3464 (0.3, 1, and 3 mg/kg) dose-dependently suppressed the METH-induced hyperlocomotion activity as the comparative example of CGS21680. Further see FIGS. 3A and 3B, it can be found that at 3 mg/kg, the administration with JMF3464 significantly lowered the METH-induced locomotion activity compared to that of the METH+vehicle group, the result of which is similar to that of the comparative example of CGS21680.

### Conditioned Place Preference (CPP)

The METH-induced conditioned place preference (CPP) task is further performed to examine the therapeutic effect of JMF3464 on METH-induced addiction.

The METH-induced CPP procedure is shown as follows. The conditioned place preference (CPP) paradigm is a standard preclinical behavioral model used to study the rewarding and aversive effects of drugs. The paradigm usually uses a two-compartment apparatus with each compartment displaying distinct contextual characteristics (e.g., wall colors/patterns and floor texture). The CPP model consisted of three phases: pre-conditioning (baseline), conditioning, and post-conditioning (i.e., CPP test). During baseline, mice were given free access to all compartments for 15 minutes one day before conditioning phase. Conditioning sessions consisted of a non-contingent (experimenter administered) injection of vehicle (control) or METH were given before placing and confining the animal in a distinct context for 30 minutes. In addition, each mouse also received either 1 (or 3) mg/kg of JMF3464 (i.p.) or vehicle 10 minutes before each METH conditioning session. Control and METH conditioning sessions occurred on the same day (separated by 4-6 h). These pairings took place over 3 days. During the conditioning session, the METH-context associations became acquired. After conditioning, time on the non-preferred side is considered as index of CPP. Lastly, one day after the conditioning sessions, mice underwent a CPP test where they were again given free access to all compartments for 15 minutes and the time spent in the METH-paired side was measured, which provided a measure of CPP expression.

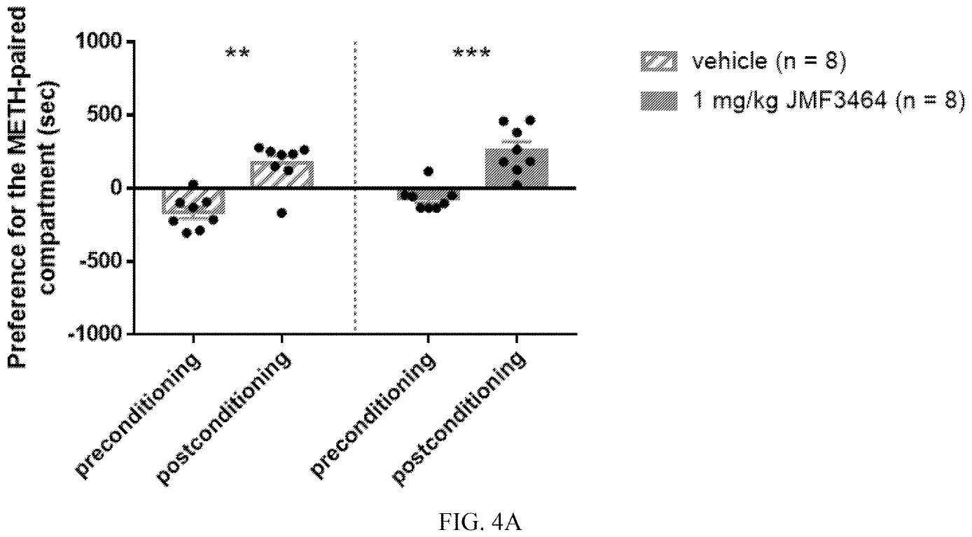

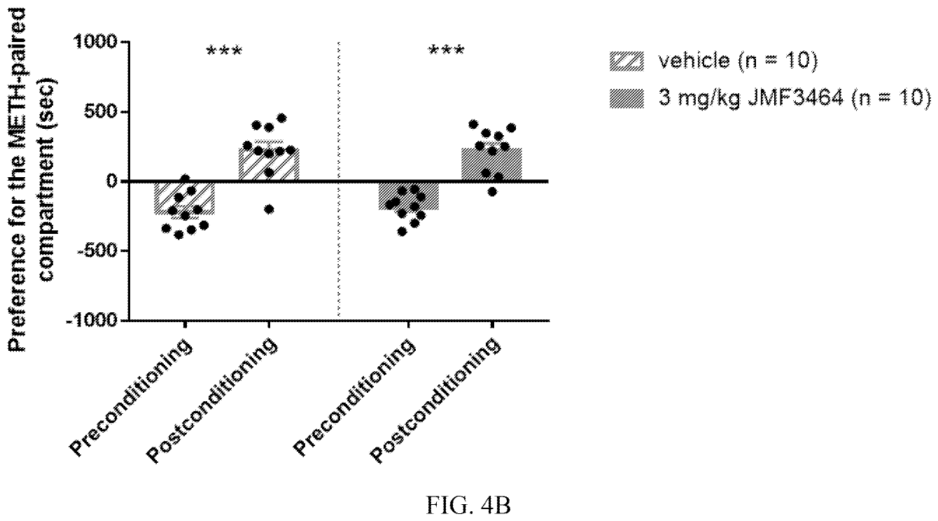

The administration of JMF3464 did not affect the METH-induced rewarding effect under current testing conditions. As shown in FIGS. 4A and 4B, neither 1 mg/kg nor 3 mg/kg of JMF3464 exerted inhibitory effect on the METH-induced CPP.

Based on the experimental results above, it can be found that JMF3464 significantly suppressed the METH-induced hyperlocomotion. Furthermore, no effect of JMF3464 on the METH-induced behavioral sensitization after repeated use or the METH-induced rewarding in CPP was observed.

### Example 2

### Evaluation Effect of Adenosine Analogue JMF1907 on Schizophrenia

Similar to the compound JMD3464, the compound of formula (III), JMF1907, represented by the following structure is an also small adenosine analogue. To assess the beneficial potential of JMF1907, the present invention determined the effect of JMF1907 in vivo using the METH-induced behavioral sensitization.

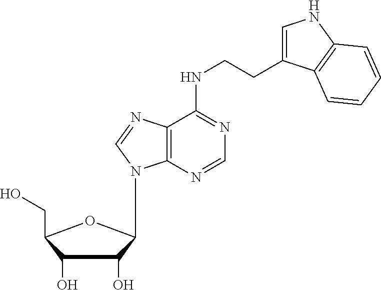

### Methods and Results

Animals: Male mice (C57BL/6; 2-3 months) were purchased from and maintained in the animal facility of National Taiwan University (Taipei, Taiwan) under standard condition with a 12-hr light/12-hr dark cycle. All animal experimental procedures were performed in accordance with the guidelines established by the IACUC of the National Taiwan University and IBMS at Academia Sinica (Taipei, Taiwan).

The locomotor activity: The locomotor activity was monitored and recorded using SMART 3.0 video tracking software (Panlab Harvard Apparatus, Barcelona, Spain) for 1 hour following the manufacture's protocol.

### METH-Induced Behavioral Sensitization for JMF1907

Similar to JMF3464 study as described above, in vivo efficacy of JMF1907 is evaluated to assess its effect on the METH-triggered responses.

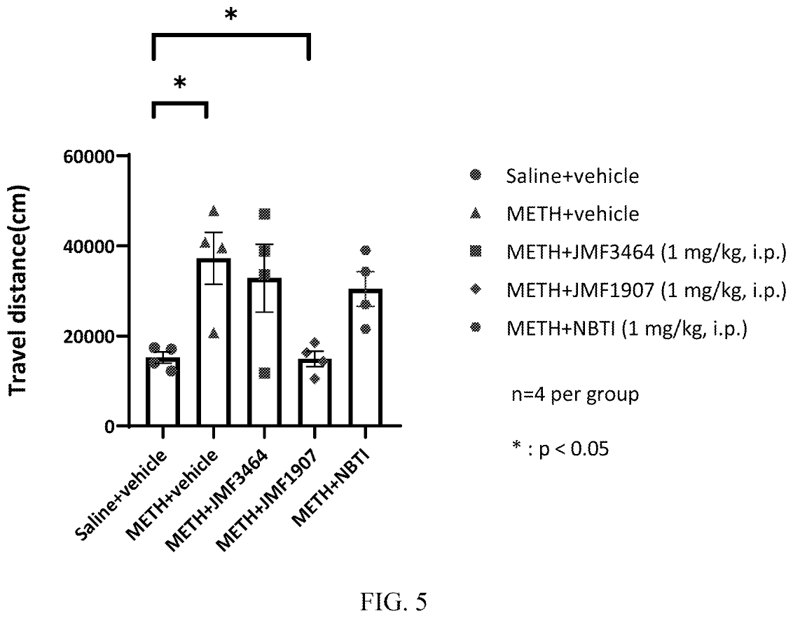

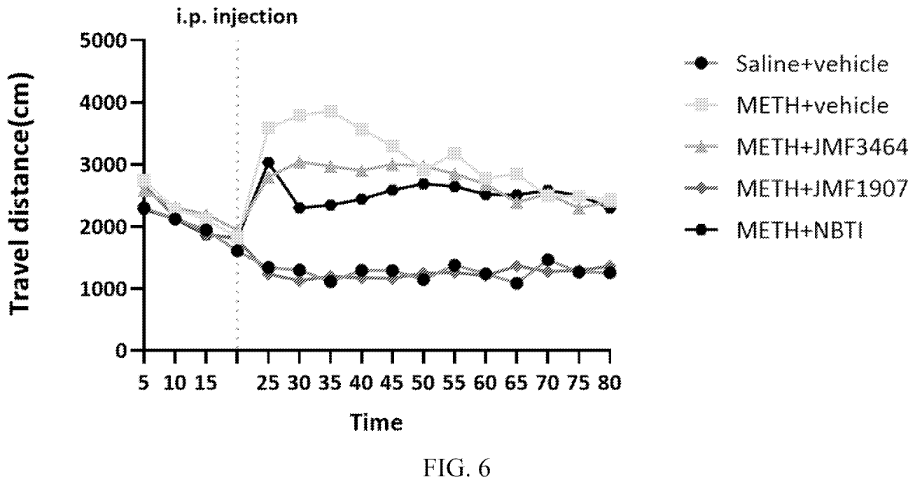

Male METH-naïve wild-type (WT) mice (3-6 months old) received intraperitoneal co-injections of JMF1907 (1 mg/kg) and METH (2 mg/kg) immediately after establishing a 20-minute baseline for locomotor activity. The locomotor activity was recorded for 60 minutes after injection. In this study, a saline+vehicle (5% DMSO) group and a METH+vehicle (5% DMSO) group are used as control groups. In addition, the compound JMF3464 described above, and the compound S-(4-Nitrobenzyl)-6-thioinosine (NBTI), known as an inhibitor of equilibrative nucleoside transporter 1 (ENTi) transporter, are used as comparative examples with the same experimental conditions as JMF1907. The resulted data was expressed and analyzed as one-way ANOVA of total travelled distance (see FIG. 5) and two-way ANOVA of distance by 5-minute time bin (see FIG. 6).

As shown in FIG. 5, in terms of overall locomotion, a 2.0 mg/kg injection of METH effectively induces hyperlocomotion activity similar to schizophrenia-like behavior. At the same dosage, JMF1907 at 1 mg/kg significantly improves METH-induced hyperactive behavior compared to the other two drugs, JMF3464 and NBTI. That is, JMF1907 injection significantly improves the behavioral performance of increased spontaneous activity induced by METH.”

Further referring to FIG. 6, when dividing the testing time into five-minute intervals, it can be observed that the five groups of mice showed no significant differences in their baseline levels of spontaneous activity prior to drug administration. However, after administering the drugs, it was observed that METH significantly induced an increase in spontaneous activity. However, compared to the other two drugs, JMF3464 and NBTI, it can be found that JMF1907 exhibited a relative improvement in METH-induced hyperactive behavior, with the efficacy of improvement being comparable to the control group (saline+vehicle). That is, in the 60-minute open field experiment, JMF1907 (1 mg/kg, i.p.) injection exhibited greater efficacy in improving METH-induced hyperactive behavior compared to the other drugs.”

Consequently, it can demonstrate that the compounds, such as JMF3464 and JMF1907, of the claimed invention is indeed able to suppress the hyperlocomotor response induced by excessive dopamine and thus has potential to treat schizophrenia.

### REFERENCES

1. Charlson, F. J., Ferrari, A. J., Santomauro, D. F., Diminic, S., Stockings, E., Scott, J. G., Mcgrath, J. J., and Whiteford, H. A. (2018). Global Epidemiology and Burden of Schizophrenia: Findings From the Global Burden of Disease Study 2016. Schizophr Bull 44, 1195-1203.
2. Perreault, M. L., Hasbi, A., O'dowd, B. F., and George, S. R. (2014). Heteromeric dopamine receptor signaling complexes: emerging neurobiology and disease relevance. Neuropsychopharmacology 39, 156-168.
3. Howes, O. D., Bose, S. K., Turkheimer, F., Valli, I., Egerton, A., Valmaggia, L. R., Murray, R. M., and Mcguire, P. (2011). Dopamine synthesis capacity before onset of psychosis: a prospective [18F]-DOPA PET imaging study. Am J Psychiatry 168, 1311-1317.
4. Murray, R. M., Bhavsar, V., Tripoli, G., and Howes, O. (2017). 30 Years on: How the Neurodevelopmental Hypothesis of Schizophrenia Morphed Into the Developmental Risk Factor Model of Psychosis. Schizophr Bull 43, 1190-1196.
5. Rampino, A., Marakhovskaia, A., Soares-Silva, T., Torretta, S., Veneziani, F., and Beaulieu, J. M. (2018). Antipsychotic Drug Responsiveness and Dopamine Receptor Signaling; Old Players and New Prospects. Front Psychiatry 9, 702.
6. Jones, C. A., Watson, D. J., and Fone, K. C. (2011). Animal models of schizophrenia. Br J Pharmacol 164, 1162-1194.

What is claimed is:

1. A method of treating schizophrenia, comprising administering to a subject in need thereof a compound of formula (I), (II) or (III): 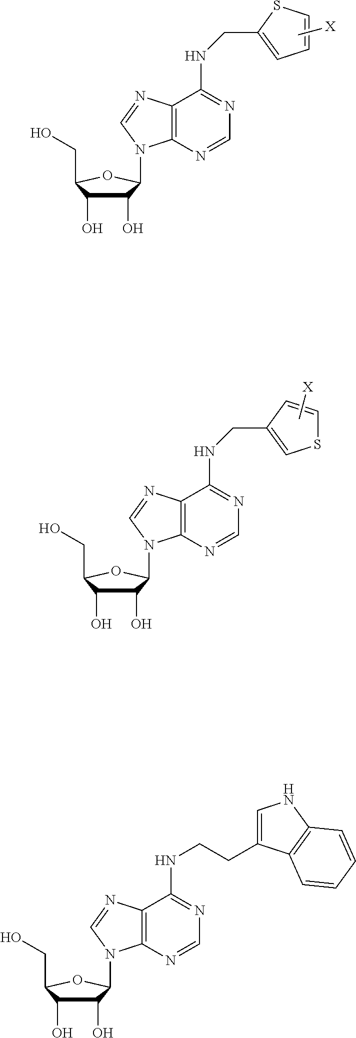
a pharmaceutically acceptable salt thereof, or a composition thereof, wherein X is halogen.

2. The method of claim 1, wherein the compound is selected from the group consisting of N⁶-[(3-halothien-2-yl)methyl]adenosine, N⁶-[(4-halothien-2-yl)methyl]adenosine, and N⁶-[(5-halothien-2-yl)methyl]adenosine.

3. The method of claim 2, wherein the compound is selected from the group consisting of N⁶-[(5-bromothien-2-yl)methyl]adenosine, N⁶-[(4-bromothien-2-yl)methyl]adenosine, N⁶-[(3-bromothien-2-yl)methyl]adenosine, N⁶-[(5-chlorothien-2-yl)methyl]adenosine, N⁶-[(4-chlorothien-2-yl)methyl]adenosine, and N⁶-[(3-chlorothien-2-yl)methyl]adenosine.

4. The method of claim 1, wherein the compound is selected from the group consisting of N⁶-[(2-halothien-3-yl)methyl]adenosine, N⁶-[(4-halothien-3-yl)methyl]adenosine, and N⁶-[(5-halothien-3-yl)methyl]adenosine.

5. The method of claim 4, wherein the compound is selected from the group consisting of N⁶-[(2-bromothien-3-yl)methyl]adenosine, N⁶-[(4-bromothien-3-yl)methyl]adenosine, N⁶-[(5-bromothien-3-yl)methyl]adenosine N⁶-[(2-chlorothien-3-yl)methyl]adenosine, N⁶-[(4-chlorothien-3-yl)methyl]adenosine, and N⁶-[(5-chlorothien-3-yl)methyl]adenosine.

6. The method of claim 1, wherein the compound, a pharmaceutically acceptable salt thereof, or a composition thereof is administered by an oral, intravenous, intramuscular, subcutaneous or intraperitoneal route.

7. The method of claim 1, wherein the composition further comprises a pharmaceutically acceptable carrier, excipient or vehicle.

8. A composition for use in a method of treating schizophrenia, comprising administering to a subject in need thereof the composition comprising a compound of formula (I), (II) or (III): 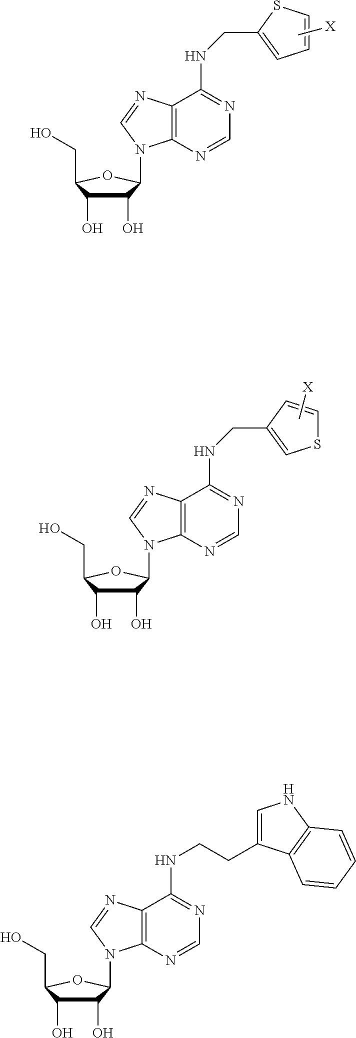
or a pharmaceutically acceptable salt thereof, wherein X is halogen.

9. The composition for use of claim 8, wherein the compound is selected from the group consisting of N⁶-[(3-halothien-2-yl)methyl]adenosine, N⁶-[(4-halothien-2-yl)methyl]adenosine, and N⁶-[(5-halothien-2-yl)methyl]adenosine.

10. The composition for use of claim 9, wherein the compound is selected from the group consisting of N⁶-[(5-bromothien-2-yl)methyl]adenosine, N⁶-[(4-bromothien-2-yl)methyl]adenosine, N⁶-[(3-bromothien-2-yl)methyl]adenosine, N⁶-[(5-chlorothien-2-yl)methyl]adenosine, N⁶-[(4-chlorothien-2-yl)methyl]adenosine, and N⁶-[(3-chlorothien-2-yl)methyl]adenosine.

11. The composition for use of claim 8, wherein the compound is selected from the group consisting of N⁶-[(2-halothien-3-yl)methyl]adenosine, N⁶-[(4-halothien-3-yl)methyl]adenosine, and N⁶-[(5-halothien-3-yl)methyl]adenosine.

12. The composition for use of claim 11, wherein the compound is selected from the group consisting of N⁶-[(2-bromothien-3-yl)methyl]adenosine, N⁶-[(4-bromothien-3-yl)methyl]adenosine, N⁶-[(5-bromothien-3-yl)methyl]adenosine N⁶-[(2-chlorothien-3-yl)methyl]adenosine, N⁶-[(4-chlorothien-3-yl)methyl]adenosine, and N⁶-[(5-chlorothien-3-yl)methyl]adenosine.

13. The composition for use of claim 8, wherein the composition is administered by an oral, intravenous, intramuscular, subcutaneous or intraperitoneal route.

14. The composition for use of claim 8, wherein the composition further comprises a pharmaceutically acceptable carrier, excipient or vehicle.
